(19) United States
(12) Patent Application Publication
(10) Pub. No.: US 2025/0059148 A1
(43) Pub. Date: Feb. 20, 2025

(54) METHOD FOR PRODUCING 5-HALOMETHYLFURFURAL AND SYSTEM THEREFOR

(71) Applicant: KOREA RESEARCH INSTITUTE OF CHEMICAL TECHNOLOGY, Daejeon (KR)

(72) Inventors: Dong Won HWANG, Daejeon (KR); In Yong EOM, Daejeon (KR); Young Kyu HWANG, Daejeon (KR); Ma Eum LEE, Daejeon (KR)

(73) Assignee: KOREA RESEARCH INSTITUTE OF CHEMICAL TECHNOLOGY, Daejeon (KR)

(21) Appl. No.: 18/701,820

(22) PCT Filed: Oct. 20, 2022
(86) PCT No.: PCT/KR2022/016002

§ 371 Date: Apr. 16, 2024

(30) Foreign Application Priority Data
Oct. 26, 2021 (KR) 10-2021-0143959

(51) Int. Cl. C07D 307/48

(52) U.S. Cl. CPC C07D 307/48

(57) ABSTRACT

According to the present disclosure, by forming normal pressure conditions in a reaction system through application of a reflux system to an aqueous phase in the presence of sulfuric acid and an inorganic salt in the dehydration reaction of biomass-derived sugar components in an open system, there is no need to include a high-pressure vessel required in a conventional 5-halomethylfurfural production process, there is no risk of safety accidents, including the explosion of a high-pressure vessel and the leakage of strong acid gas, and there is a remarkable effect in that yield is improved three-fold or more compared to the case of using a conventionally used acid catalyst under the normal pressure conditions.

### TECHNICAL FIELD

The present disclosure relates to a method and system for producing 5-halomethylfurfural and, more particularly, to a method and system for economically and environment-friendlily producing 5-halomethylfurfural by using normal pressure in a reflux system rather than using high pressure in a mixed two-phase system of an aqueous phase and an organic phase.

### BACKGROUND ART

The increasing global demand for fuel and chemical raw materials has led to a rapid increase in the use of fossil fuels such as coal and petroleum, and the emission of carbon dioxide resulting from the use of fossil fuels is causing global warming and extreme weather. For this reason, technologies to use sustainable resources alternative to fossil fuels are being researched and developed.

For example, there has been an attempt to make fuels and chemicals from biomass, which is one example of sustainable resources, including cellulose, hemicellulose, lignin, and starch.

One example of biomass-derived fuel is bioethanol, which is produced by saccharifying crops, such as barley and corn, or plants, such as fiber-containing trees and straw, and fermenting them using microorganisms. In addition, biodiesel, which is another example, is produced by extracting oil from crops, such as rapeseed and soybeans and esterifying the oil.

However, since the crops used for the production of bioethanol are food, when they are used to secure economics and meet large-scale demand, there are problems such as difficulty in securing land for mass cultivation and unstable harvest due to climate change. Therefore, technologies to utilize non-food plants, such as straw left over after crop harvesting and wood, are under being researched and developed because the non-food plants are abundant resources.

5-Hydroxymethylfurfural (HMF), which is one of the value-added chemical raw materials derived from waste wood, has little use. However, 2,5-furandicarboxylic acid, which is a major HMF derivative, can replace terephthalic acid used for the production of polyester, and 5-dimethylfuran is a promising value-added chemical raw material that can be used as a biofuel because it has a higher energy density than bioethanol.

HMF is an intermediate produced in the process of producing levulinic acid through acid-catalyzed dehydration of C₆-sugars. However, when hemicellulose including xylose is used as a raw material, a biological conversion process using microorganisms is required, and commercialization is difficult due to low speed, yield, and stability.

Under these circumstances, U.S. Pat. No. 7,829,732 (issued Nov. 9, 2010) discloses a technique for pretreating and dehydrating biomass, including cellulose and hemicellulose, to obtain 5-chloromethylfurfural (CMF). Specifically, the document describes the following method: an organic solvent, which is an extraction solvent, is introduced into a reaction system to create a two-phase system composed of an aqueous phase and an organic phase; 5-chloromethylfurfural (CMF) is produced from biomass in the presence of an acid catalyst and chlorine ions in the aqueous phase; and the produced 5-chloromethylfurfural (CMF) is moved from the aqueous phase to the organic phase and recovered in the organic phase. However, in the case of using hydrochloric acid (HCl), which is described in the document as an acid catalyst, not only does it require an autoclave facility, but it can also cause safety issues such as explosions and leakage of hydrochloric acid gas during the production of 5-chloromethylfurfural (CMF), which is a type of 5-halomethylfurfural. Thus, there is a need for a 5-halomethylfurfural production method providing a high yield without using a high-pressure vessel unlike compared to conventional methods using an acid catalyst.

### DISCLOSURE

### Technical Problem

The objective of the present disclosure is to provide a high-yield production method for producing 5-halomethylfurfural, the method being capable of causing a dehydration reaction of biomass-derived sugar at normal pressure conditions in an open system, thereby not requiring a high-pressure vessel.

### Technical Solution

To solve the above problems, the present disclosure provides a method of producing 5-halomethylfurfura, the method including: (a) preparing a mixed aqueous solution containing a sugar component, a sulfuric acid aqueous solution, and an inorganic salt comprising a halogen in a reactor; (b) adding toluene as an extraction organic solvent to the mixed aqueous solution to obtain a mixed two-phase solution of an aqueous phase and an organic phase; (c) stirring the mixed two-phase solution to homogeneously mix the aqueous phase and the organic phase, and refluxing the solvent while raising the temperature of the mixed two-phase solution to induce a dehydration reaction; and (d) rendering the mixed two-phase solution stationary after the step (c) to partition into the aqueous phase and the organic phase, and recovering 5-halomethylfurfural from the organic phase.

In one embodiment of the present disclosure, the sugar component of the step (a) may include one or more components selected from: monosaccharides including glucose, fructose, galactose; disaccharides comprising maltose, sucrose, lactose; and polysaccharides including cellulose, hemicellulose, and starch comprising hexose.

In one embodiment of the present disclosure, the concentration of sulfuric acid in the step (a) may be in a range of from 40 to 70 wt % with respect to the total weight of the sulfuric acid aqueous solution.

In one embodiment of the present disclosure, in the mixed two-phase solution of the step (b), a volume ratio of the organic phase to the aqueous phase may be in a range of from 2 to 10.

In one embodiment of the present disclosure, the mixed two-phase solution in the step (c) may be raised to a temperature in a range of 80° C. to 111° C.

In one embodiment of the present invention, the sugar component may be derived from biomass.

The disclosure also provides a method for producing 5-hydroxymethyl furfural (HMF) from the 5-halomethylfurfural obtained by the method described above.

### Advantageous Effects

According to the present disclosure, normal pressure conditions are formed in a reaction system through application of a reflux system to an aqueous phase during a dehydration reaction of biomass-derived sugar components in an open system. Therefore, the method of the present disclosure does not require the use of a high-pressure vessel unlike a conventional 5-halomethylfurfural production process, thereby being capable of avoiding risk of safety accidents, including the explosion of the high-pressure vessel and the leakage of acid gas, implementing an economic process, and having a remarkable effect in improving yield three-fold or more compared to the case of using a conventionally used acid catalyst under normal pressure conditions.

### DESCRIPTION OF DRAWINGS

FIG. 1 is a diagram illustrating a mechanism by which HMF generated from a dehydration reaction of hexose in an aqueous phase is distributed to an organic phase; and

FIG. 2 is a graph of the CMF yield over time as a function of the type of acid catalyst and the presence/absence of NaCl.

### BEST MODE

Unless otherwise defined, all terms including technical and scientific terms used herein have the same meaning as commonly understood by those who are ordinarily skilled in the art to which the present disclosure pertains. In general, the nomenclature used herein is that well known and commonly used in the art.

It will be further understood that the terms “comprise”, “include”, or “have”, when used in this specification, specify the presence of an element, but do not preclude the presence or addition of one or more other elements unless the context clearly indicates otherwise.

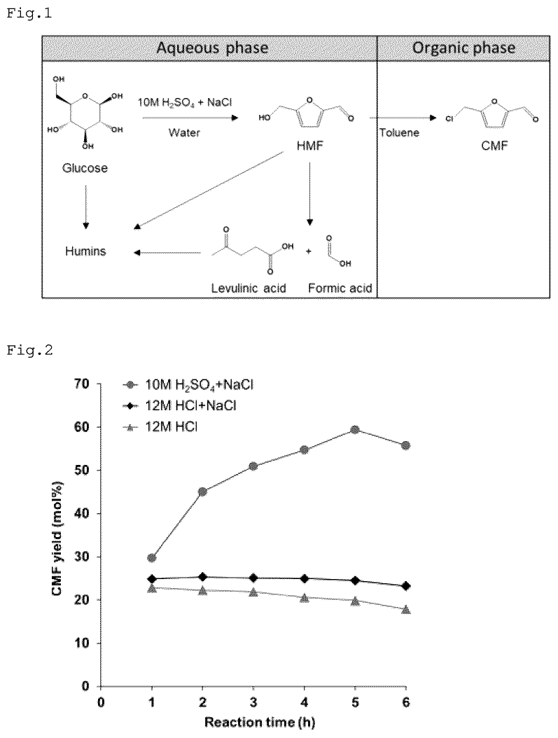

FIG. 1 is a diagram illustrating a mechanism by which HMF generated from a dehydration reaction of hexose in an aqueous phase is distributed to an organic phase.

Referring to FIG. 1, D-glucose is converted to HMF by successive dehydration reactions. When the dehydration of HMF goes further than the illustrated state, levulinic acid is generated. However, since HMF has a wider range of industrial applications than levulinic acid, HMF is in greater demand. Therefore, as a method for preventing the conversion of HMF to levulinic acid and selectively obtaining HMF, there is conventionally known a technique that indirectly obtains 5-chloromethylfurfural (CMF). According to the technique, hydroxyl groups (—OH) in HMF are substituted with halogen atoms so that the HMF is converted to 5-chloromethylfurfural (CMF), and the CMF is distributed in an extraction solvent.

The reaction to produce 5-halomethylfurfural from a sugar component such as glucose proceeds through dehydration and chlorination of sugar in a concentrated hydrochloric acid aqueous solution (12 M, 37 wt %). When the hydrochloric acid aqueous solution is heated to a temperature of 108.6° C. under atmospheric pressure, and the HCl concentration of the aqueous solution is 20.2 wt % under atmospheric pressure, azeotrope of HCl/water is formed. Therefore, the concentration of hydrochloric acid cannot be increased without using an autoclave reactor. That is, it was difficult to perform the reaction with a conventional reactor due to low yield and the risk of safety accidents including leakage of hydrochloric acid gas. To solve these problems, the applicant conceived a high-yield method for producing 5-halomethylfurfural through a dehydration reaction of a sugar component by using sulfuric acid (H₂SO₄) as an acid catalyst to replace HCl and using a halogen-providing inorganic salt for conversion to 5-halomethylfurfural, without using an autoclave.

Hereinafter, the method of producing 5-halomethylfurfural using sulfuric acid under reflux conditions, according to the present disclosure, will be described in detail.

The method of producing 5-halomethylfurfural from a sugar component, according to the present disclosure, includes: (a) preparing a mixed aqueous solution containing a sugar component, a sulfuric acid aqueous solution, and an inorganic salt comprising a halogen in a reactor; (b) adding toluene as an extraction organic solvent to the mixed aqueous solution to obtain a mixed two-phase solution of an aqueous phase and an organic phase; (c) stirring the mixed two-phase solution to homogeneously mix the aqueous phase and the organic phase, and refluxing the solvent while raising the temperature of the mixed two-phase solution to induce a dehydration reaction; and (d) rendering the mixed two-phase solution stationary after the step (c) to partition into the aqueous phase and the organic phase, and recovering 5-halomethylfurfural from the organic phase.

The sugar component of step (a) above is a raw material component that can be converted to HMF, and the concentration thereof may range from 0.01 to 20 wt %. The sugar component may include at least one selected from: monosaccharides including glucose, fructose, and galactose; disaccharides including maltose, sucrose, and lactose; and polysaccharides including cellulose, hemicellulose, and starch including hexoses. Preferably, the sugar component may include glucose, cellulose, or both.

The sulfuric acid aqueous solution in step (a) above is non-volatile with almost no sulfuric acid present in gas phase until the concentration of H₂SO₄ becomes 70 wt %. Even in the case of highly concentrated sulfuric acid with a concentration of 98 wt %, the concentration of H₂SO₄ in gas phase remains lower than that in liquid phase. Therefore, the reaction for the production of 5-halomethylfurfural from sugars can be easily carried with a simple solvent reflux system without requiring an autoclave. That is, it is possible to easily and highly efficiently produce 5-halomethylfurfural with ordinary equipment without using special temperature control systems.

The concentration of the sulfuric acid ranges from 10 to 70 wt % with respect to the total weight of the sulfuric acid aqueous solution and preferably ranges from 40 to 70 wt %. When the concentration of sulfuric acid is lower than 10 wt %, there is a problem that it is not effective in promoting the dehydration reaction as an acid catalyst. When the concentration of sulfuric acid exceeds 70 wt %, there is a problem that safety accidents may occur because sulfuric acid may be present in gas phase.

The halogen-containing inorganic salt used in step (a) above is a halogen source for substituting HMF. It is used because a conventional acid catalyst, i.e., hydrochloric acid, is replaced with sulfuric acid. The inorganic salt, which is an ionic substance, increases the hydrophilicity of the aqueous phase in the mixed two-phase solution, thereby promoting the dispersion of 5-halomethylfurfural in the organic phase, so that the yield can be improved. The halogen-containing inorganic salt may include a metal ion, and the metal ion may form a salt with sulfate ions (SO₄²⁻), thereby cause a salting-out effect, which further promotes the dispersion of 5-halomethylfurfural.

Any halogen-containing inorganic salt can be used without limitation as long as it can dissolve to provide a halogen ion (X^{-═}F⁻, Cl⁻, Br⁻, or I⁻). For example, a salt containing a metal cation and a chlorine anion can be used. Specifically, at least one alkaline metal chloride selected from NaCl, LiCl, KCl, RbCl, and CsCl; at least one alkaline earth metal chloride selected from MgCl₂, CaCl₂), SrCl₂, and BaCl₂; at least one alkaline metal bromide selected from NaBr, LiBr, KBr, RbBr, and CsBr; at least one alkaline earth metal bromide of MgBr₂, CaBr₂, SrBr₂, and BaBr₂; at least one alkaline metal iodide selected from NaI, LiI, KI, RbI, and CsI; at least one alkaline earth metal iodide selected from MgI₂, CaI₂, SrI₂, and BaI₂; at least one alkaline metal fluoride selected from NaF, LiF, KF, RbF, and CsF; and/or at least one alkaline earth metal fluoride selected from MgF₂, CaF₂, SrF₂, and BaF₂ may be used. Preferably, at least one alkaline metal chloride selected from LiCl, KCl, RbCl, and CsCl and/or at least one alkaline earth metal chloride MgCl₂, CaCl₂, SrCl₂, and BaCl₂ may be used. More preferably, NaCl may be used.

In the mixed two-phase solution in step (b), a volume ratio of the organic phase to the aqueous phase may be in a range of from 2 to 10.

In step (c) above, the mixed two-phase solution may be heated to a range of 80° C. and 111° C. When the raised temperature is lower than 80° C., there is a problem that the dehydration reaction rate is excessively slow, and the reaction temperature may be limited because the solution is not raised to above the boiling point of the solvent (111° C. for toluene) under normal pressure reflux conditions.

Hereinafter, with reference to the accompanying drawings, a method for the production of 5-halomethylfurfural using sulfuric acid under refluxing conditions according to the present disclosure will be described in detail through experimental examples.

### Experimental Example 1

0.60 g of glucose (99%, Sigma) and 1.87 g of NaCl (extra pure, DC Chemical) equivalent to 0.6 moles of sulfuric acid added to 6 mL (7.80 g) of 10 M sulfuric acid aqueous solution were placed in a 1-necked flask, 30 mL of toluene (99%, Samchun) was added thereto, and a condenser in which 0° C. ethanol was circulating was installed to enable reflux. The internal temperature of the flask was raised to 111° C., which is the boiling point of toluene, while the mixed two-phase solution in the flask was stirred at 1400 rpm, and the dehydration reaction and reflux were carried out under normal pressure. After 300 minutes of the reaction, the flask was put in not a 0° C. cold water tank for quenching so that the toluene layer was separated from the water layer. Anhydrous magnesium sulfate was added to the toluene layer to remove moisture, and the remainder was filtered to obtain toluene. The obtained toluene was subjected to reduced-pressure distillation (room temperature, <50 mmHg) to recover a non-volatile 5-chloromethylfurfural (CMF) crude product. The recovered 5-chloromethylfurfural (CMF) crude product was subjected to column chromatography (silica gel, CH₂Cl₂:Et₂O, 2:1) to finally recover high-purity 5-chloromethylfurfural (CMF). For the quantitative analysis of 5-chloromethylfurfural (CMF) generated in the toluene layer, the 5-chloromethylfurfural (CMF) generated in the toluene layer was quantified by using a response factor measured between the separated high-purity 5-chloromethylfurfural and 1-heptane as an internal standard with the use of a DB-624UI column and a GC analyzer equipped with FID.

In addition to the separated 5-chloromethylfurfural (CMF), the amount of unreacted glucose dissolved in the water layer was analyzed with HPLC equipped with an Aminex HPX-87H column and a refractive index detector, in which 5 mM sulfuric acid aqueous solution was used as a mobile phase (at a flow rate of 0.6 ml/min), and the reaction was performed for 1 to 6 hours. Table 1 below shows the glucose conversion rate and 5-chloromethylfurfural (CMF) selectivity under conditions in which the maximum 5-chloromethylfurfural (CMF) yield was obtained.

### Experimental Examples 2 to 12

5-Chloromethylfurfural (CMF) was produced in the same manner as in Example 1 except that 1 M to 12 M sulfuric acid aqueous solutions were used instead of 10 M sulfuric acid aqueous solution so as to achieve the sulfuric acid molarity shown in the table below.

### Experimental Examples 13 to 21

5-Chloromethylfurfural (CMF) was produced in the same manner as in Example 1 above, except that the number of moles of NaCl in Example 1 was adjusted to be as shown in Table 1 below.

### Experimental Example 22

5-Chloromethylfurfural (CMF) was produced in the same manner as in Example 1 above, except that sulfuric acid aqueous solution was not used.

### Experimental Example 23

5-Chloromethylfurfural (CMF) was produced in the same manner as in Example 1 above, except that NaCl was not used.

### Experimental Example 24

5-Chloromethylfurfural (CMF) was produced in the same manner as in Example 1 above, except that 12M HCl was used instead of 10M sulfuric acid aqueous solution.

### Experimental Example 25

5-Chloromethylfurfural (CMF) was produced in the same manner as in Example 24 above, except that NaCl was not used.

TABLE 1

| | | | | | | Conv. | | |
|---|---|---|---|---|---|---|---|---|
| | | | | | | Of | Sel. of | Yield of |
| | glucose | HCl | H₂SO₄ | NaCl | Toluene | glucose | 5-CMF | 5-CMF |
| | (mmol) | (mol) | (mol) | (mol) | (mL) | (%) | (%) | (%) |
| Ex. 1 | 0.0034 | — | 0.053 | 0.03 | 30 | 98.2 | 60.5 | 59.4 |
| Ex. 2 | 0.0033 | — | 0.005 | 0.03 | 30 | 13.5 | 4.2 | 0.6 |
| Ex. 3 | 0.0033 | — | 0.010 | 0.03 | 30 | 20.8 | 7.4 | 1.5 |
| Ex. 4 | 0.0032 | — | 0.016 | 0.03 | 30 | 29.5 | 12.4 | 3.7 |
| Ex. 5 | 0.0033 | — | 0.021 | 0.03 | 30 | 32.7 | 16.5 | 5.4 |
| Ex. 6 | 0.0033 | — | 0.026 | 0.03 | 30 | 37.9 | 29.5 | 11.2 |
| Ex. 7 | 0.0032 | — | 0.032 | 0.03 | 30 | 51.3 | 34.2 | 17.5 |
| Ex. 8 | 0.0033 | — | 0.037 | 0.03 | 30 | 64.1 | 39.8 | 25.5 |
| Ex. 9 | 0.0033 | — | 0.043 | 0.03 | 30 | 87.7 | 41.5 | 36.4 |
| Ex. 10 | 0.0033 | — | 0.048 | 0.03 | 30 | 90.7 | 43.5 | 39.5 |
| Ex. 11 | 0.0032 | — | 0.059 | 0.03 | 30 | 98.9 | 56.4 | 55.8 |
| Ex. 12 | 0.0033 | — | 0.064 | 0.03 | 30 | 99.0 | 54.2 | 53.7 |
| Ex. 13 | 0.0035 | — | 0.054 | 0.01 | 30 | 99.3 | 41.4 | 41.1 |
| Ex. 14 | 0.0034 | — | 0.054 | 0.02 | 30 | 99.0 | 56.0 | 55.5 |
| Ex. 15 | 0.0034 | — | 0.053 | 0.04 | 30 | 98.0 | 56.6 | 55.4 |
| Ex. 16 | 0.0034 | — | 0.053 | 0.05 | 30 | 97.9 | 53.8 | 52.6 |
| Ex. 17 | 0.0034 | — | 0.053 | 0.06 | 30 | 97.8 | 53.3 | 52.1 |
| Ex. 18 | 0.0034 | — | 0.053 | 0.07 | 30 | 97.8 | 53.4 | 52.2 |
| Ex. 19 | 0.0034 | — | 0.053 | 0.08 | 30 | 97.5 | 52.7 | 51.4 |
| Ex. 20 | 0.0034 | — | 0.053 | 0.09 | 30 | 97.3 | 52.4 | 51.0 |
| Ex. 21 | 0.0034 | — | 0.053 | 0.1 | 30 | 97.3 | 51.8 | 50.4 |
| Ex. 22 | 0.0033 | — | — | 0.03 | 30 | 1.3 | 0.0 | 0 |
| Ex. 23 | 0.0034 | — | 0.053 | — | 30 | 98.7 | 0 | 0 |
| Ex. 24 | 0.0030 | 0.055 | — | 0.03 | 30 | 97.1 | 26.1 | 25.3 |
| Ex. 25 | 0.0030 | 0.055 | — | — | 30 | 98.5 | 23.2 | 22.9 |

Table 1 above shows the results of experiments with different combinations or amounts of hydrochloric acid, sulfuric acid, and NaCl in the production of 5-chloromethylfurfural (CMF), and FIG. 2 2 shows the yield of CMF as a function of time for the case of using sulfuric acid and NaCl, the case of using hydrochloric acid and NaCl, and the case of using only hydrochloric acid.

Comparing Experimental Example 1 with Experimental Examples 22 and 23 in Table 1 above, it is seen that both sulfuric acid and NaCl are required in the reaction to produce 5-chloromethylfurfural (CMF), as no 5-chloromethylfurfural (CMF) was produced in Experimental Example 22 using sulfuric acid alone or in Experimental Example 23 using NaCl alone.

In addition, Experimental Example 24 and Experimental Example 25 were performed using hydrochloric acid instead of sulfuric acid. In the case of using hydrochloric acid but not using NaCl as in Experimental Example 25, it is seen that the yield of 5-chloromethylfurfural (CMF) was 22.9%. That is, Example 25 appears more favorable in terms of 5-chloromethylfurfural (CMF) yield than Experimental Example 23 using sulfuric acid alone without NaCl.

Experimental Examples 24 and 25 are cases in which NaCl was not added to hydrochloric acid. Experimental Example 24 in which NaCl was not added shows a slight decrease in glucose conversion compared to Experimental Example 25 in which NaCl was not added, but shows an increase in the selectivity of 5-chloromethylfurfural (CMF), thereby showing an overall increase in the yield of 5-chloromethylfurfural (CMF).

However, the yield of 5-chloromethylfurfural (CMF) is only 25.3% at most when hydrochloric acid is used alone or when hydrochloric acid and NaCl are used together. That is, the yield is about 0.43 times less than the yield (59.4%) of Experimental Example 1 in which sulfuric acid and NaCl are used in combination.

Accordingly, it is found that for the production of CMF from biomass in an open system with reflux of a solvent as in the work suggested in the present application, a combination of sulfuric acid and NaCl can be used to obtain much higher yields.

Experimental Examples 2 through 21 adjusted the molarity of sulfuric acid and NaCl to show the appropriate molarities.

Referring to FIG. 2, in the conversion of glucose to 5-chloromethylfurfural (CMF), when sulfuric acid and NaCl are used in combination, the yield of CMF increases with the reaction time until the reaction time increases to 5 hours, and then the yield of CMF tends to decrease when the reaction time is longer than 5 hours. In the case of using hydrochloric acid and NaCl, there is no change in the yield of CMF even though the reaction time increases. In the case of using hydrochloric acid alone, the yield tends to decrease slightly with the increase of the reaction time, showing that there are many differences in the reactivity depending on the type of acid used in the conversion process of CMF.

The exemplary embodiments described herein and the configurations illustrated in the drawings are presented for illustrative purposes and do not exhaustively represent the technical spirit of the present invention it should be appreciated that there will be various equivalents and modifications that can replace the exemplary embodiments and the configurations at the time at which the present application is filed.

1. A method of producing 5-halomethylfurfural (CMF) from a sugar component, the method comprising:
(a) preparing a mixed aqueous solution comprising a sugar component, a sulfuric acid aqueous solution, and a halogen-containing inorganic salt in a reactor;
(b) adding toluene as an extraction organic solvent to the mixed aqueous solution to obtain a mixed two-phase solution of an aqueous phase and an organic phase;
(c) stirring the mixed two-phase solution to homogeneously mix the aqueous phase and the organic phase, and refluxing the solvent while raising the temperature of the mixed two-phase solution to induce a dehydration reaction; and
(d) rendering the mixed two-phase solution stationary after the step (c) to partition into the aqueous phase and the organic phase, and recovering 5-halomethylfurfural from the organic phase.

2. The method of claim 1, wherein the sugar component of the step (a) comprises one or more components selected from: monosaccharides comprising glucose, fructose, galactose; disaccharides comprising maltose, sucrose, lactose; polysaccharides comprising cellulose, hemicellulose, and starch comprising hexose.

3. The method of claim 1, wherein the concentration of sulfuric acid in the step (a) is from 40 to 70 wt % with respect to the total weight of the sulfuric acid aqueous solution.

4. The method of claim 1, wherein the halogen-containing inorganic salt in the step (a) is at least one selected from: at least one alkaline metal chloride selected from NaCl, LiCl, KCl, RbCl, and CsCl; at least one alkaline earth metal chloride selected from MgCl₂, CaCl₂, SrCl₂, and BaCl₂; at least one alkaline metal bromide selected from NaBr, LiBr, KBr, RbBr, and CsBr; at least one alkaline earth metal bromide selected from MgBr₂, CaBr₂, SrBr₂, and BaBr₂; at least one alkaline metal iodide selected from NaI, LiI, KI, RbI, and CsI; at least one alkaline earth metal iodide selected from MgI₂, CaI₂, SrI₂, and BaI₂; at least one alkaline metal fluoride selected from NaF, LiF, KF, RbF, and CsF; and at least one alkaline earth metal fluoride selected from MgF₂, CaF₂, SrF₂, and BaF₂.

5. The method of claim 1, wherein in the mixed two-phase solution in the step (b), a volume ratio of the organic phase to the aqueous phase is in a range of from 2 to 10.

6. The method of claim 1, wherein the mixed two-phase solution in step (c) is raised to a temperature in a range of 80° C. to 111° C.

7. The method of claim 1, wherein the said sugar component is derived from biomass.

8. A method of producing 5-hydroxymethylfurfural (HMF) using the 5-halomethylfurfural prepared by the method of claim 1.
